(19) United States
(12) Patent Application Publication
(10) Pub. No.: US 2022/0175271 A1
(43) Pub. Date: Jun. 9, 2022

(54) WEIGHT LOSS DETECTION AND MONITORING SYSTEM

(71) Applicant: Breathefit, LLC, Palm Beach, CA (US)

(72) Inventors: Vincent Battaglia, Palm Desert, CA (US); Ruben Meerman, Mermaid Beach (AU)

(73) Assignee: Breathefit, LLC, Palm Desert, CA (US)

(21) Appl. No.: 17/540,464

(22) Filed: Dec. 2, 2021

Related U.S. Application Data
(60) Provisional application No. 63/121,810, filed on Dec. 4, 2020.

(51) Int. Cl. A61B 5/083; A61B 5/091; A61B 5/097; A61B 5/00; G01N 21/61

(52) U.S. Cl. CPC A61B 5/0836; A61B 5/091; A61B 5/097; A61B 5/6804; A61B 2562/0247; G01N 21/61; A61B 2560/0223; A61B 2562/0271; A61B 5/7246

(57) ABSTRACT

A system for measuring a mass of exhaled carbon for weight loss monitoring. The system includes a capture device for capturing exhaled breath and determining a mass of carbon in the exhaled breath, a respiratory inductive plethysmograph (RIP) device incorporated into apparel, and a processor incorporated into a computing device. Mass of exhaled carbon may be determined by the capture device and computing device alone. Alternatively, mass of exhaled carbon may be determined by the RIP device and the computing device alone, after calibration of the RIP device using the capture device.

### PRIORITY DATA

This application claims priority to U.S. Provisional Patent Application No. 63/121,810 filed on Dec. 4, 2020 entitled “WEIGHT LOSS DETECTION AND MONITORING SYSTEM”, which application is incorporated by reference herein in its entirety.

### BACKGROUND

General estimates put two-thirds of Americans as overweight, and one-third as obese. Given these numbers, a wide variety of weight loss and monitoring systems exist. Some people monitor their weight using a household scale, while others employ a more exacting monitoring system of counting their intake and burn of calories. The term “burning” calories is associated with a popular misconception, even among health professionals, that fat is converted into energy during weight loss (Meerman et al., “When somebody loses weight, where does the fat go?,” British Medical Journal, 349:g7257 (2014), hereinafter “Meerman”). This is incorrect. The mass of fat is converted primarily to carbon dioxide and the remainder becomes water. It would be helpful to develop a new system to monitor and motivate weight loss by measuring exhaled carbon dioxide.

### BRIEF DESCRIPTION OF THE DRAWINGS

FIG. 1 is an overview of the various components of an embodiment of the present technology.

FIG. 2 is view of a capture device and computing system according to an embodiment of the present technology.

FIG. 3 is a more detailed view of the capture device and computing system according to an embodiment of the present technology.

FIG. 4 is a flowchart illustrating the operation of a mass computation software algorithm of the present technology to determine a mass of carbon expelled during exhalation.

FIG. 5 is view of a RIP device and computing system according to an embodiment of the present technology.

FIG. 6 is a schematic block diagram of components of a RIP device according to embodiments of the present technology.

FIG. 7 is a flowchart illustrating operation of a software algorithm for calibrating the RIP device using the capture device according to embodiments of the present technology.

FIG. 8 is an illustration of a user interface according to embodiments of the present technology.

FIG. 9 is a schematic block diagram of a computing environment according to an embodiment of the present technology.

FIGS. 10-13 are views of a user wearing different configurations of straps of a RIP device according to embodiments of the present technology.

### DETAILED DESCRIPTION

Embodiments of the present technology will now be described with reference to the figures, which in general relate to a system for measuring a mass of exhaled carbon for weight loss monitoring. The system comprises a capture device for capturing exhaled breath and determining a mass of carbon in the exhaled breath, a respiratory inductive plethysmograph (RIP) or other respiratory volume sensor (RVS) device incorporated into apparel, and a processor incorporated into a smart phone, smart watch or other computing device. Mass of exhaled carbon may be determined by the capture device and computing device alone. Alternatively, mass of exhaled carbon may be determined by the RSV device and the computing device alone, after calibration of the RIP device using the capture device.

The capture device measures parameters of the exhaled carbon dioxide including a volume of the exhaled breath and a percentage of carbon dioxide in the exhaled breath. The capture device includes a mask or mouthpiece directly capturing a volume of exhaled air as the air flows through the mask or mouthpiece to the surrounding environment. The capture device further includes a spirometer for measuring the volume, and a carbon dioxide sensor for measuring a concentration of carbon dioxide in the exhaled air. This information is transmitted to the computing system which then determines a mass of carbon in the exhaled air. The capture device may further be used to calibrate the RSV device. Thereafter, the RSV device may be used without the mask or mouthpiece capture device to measure the volume of air breathed, which again may be transmitted to the computing device for determination of an amount of carbon exhaled by mass.

It is understood that the present invention may be embodied in many different forms and should not be construed as being limited to the embodiments set forth herein. Rather, these embodiments are provided so that this disclosure will be thorough and complete and will fully convey the invention to those skilled in the art. Indeed, the invention is intended to cover alternatives, modifications and equivalents of these embodiments, which are included within the scope and spirit of the invention as defined by the appended claims. Furthermore, in the following detailed description of the present invention, numerous specific details are set forth in order to provide a thorough understanding of the present invention. However, it will be clear to those of ordinary skill in the art that the present invention may be practiced without such specific details.

The terms “top” and “bottom,” “upper” and “lower” and “vertical” and “horizontal” as may be used herein are by way of example and illustrative purposes only, and are not meant to limit the description of the invention inasmuch as the referenced item can be exchanged in position and orientation. Also, as used herein, the terms “substantially” and/or “about” mean that the specified dimension or parameter may be varied within an acceptable manufacturing tolerance for a given application. In one embodiment, the acceptable manufacturing tolerance is ±2.5%.

Three of the four macronutrients, carbohydrates, fat and alcohol, consist of the three elements, carbon, hydrogen and oxygen, and are all metabolised to carbon dioxide and water with the following stoichiometries:

### Carbohydrates (Glucose):

C₆H₁₂O₆+6O₂---->6CO₂+6H₂O

### Fat (Average Human Triglyceride):

C₅₅H₁₀₄O₆+78O₂---->55CO₂+52H₂O

### Alcohol (Ethanol):

C₂H₆O+3O₂---->2CO₂+3H₂O

The fourth macronutrient, protein, is a polymer made of amino acids, which also consists of carbon, hydrogen and oxygen atoms plus the two additional elements, nitrogen and sulfur. Standard human protein (also known as Kleiber's protein) has the chemical formula C₁₀₀H₁₅₉N₂₆O₃₂S_{0.7}. This stoichiometry reveals that 87% of the carbon atoms in human protein are metabolised to carbon dioxide and the remaining 13% combine with nitrogen to form urea. The sulfur atoms are metabolised to sulfuric acid and excreted in urine as sulfate, such that the total oxidation of human protein may be summarised by the following equation:

C₁₀₀H₁₅₉N₂₆O₃₂S_{0.7}+105.3O₂---->87CO₂+13CH₄N₂O+52.8H₂O+0.7H₂SO₄

When more carbon atoms are consumed in the diet than exhaled by the lungs, the excess carbon atoms are stored in the body as glycogen (a polymerised form of glucose) in the liver, muscles and other tissues, and as triglycerides stored in adipose tissue (fat).

The human body cannot store excess protein but instead converts the amino acids derived from digested protein to glucose or ketones which can then be further metabolised according to needs.

When less carbon atoms are consumed in the diet than exhaled by the lungs, the shortfall is met by carbon atoms obtained from glycogen and triglycerides stored within the body.

The mass of the glycogen and triglyceride molecules stored in the body is therefore not converted to energy during weight loss, as is widely believed, but rather to carbon dioxide and water, according to the formulas outlined above. While these formulas show that both glycogen and fat molecules are lost as carbon dioxide molecules and water molecules, they do not reveal the ratios of the mass of carbon dioxide compared to the mass of the water produced by their oxidation. Meerman used the results of stable isotope studies to determine that 84% of the mass of fat molecules is exhaled as carbon dioxide, and the remaining 16% is lost as water, primarily through perspiration, urine and feces. Thus, for example, 10 kg of fat (C55H10406) are lost from the body as 8.4 kg exhaled carbon dioxide and 1.6 kg of exuded water. Using the same technique for glucose, it can be shown that 75% of the mass of glycogen is exhaled as carbon dioxide and the remaining 25% is converted to water. Therefore, for weight loss to occur, the vast majority of the mass stored in the body's glycogen and fat deposits must be exhaled as carbon dioxide.

It is not physiologically possible to accelerate weight loss by simply hyperventilating in order to exhale more carbon dioxide molecules at rest. If carbon dioxide exhalation exceeds carbon dioxide production, the body instead becomes hypocapnic, with a constellation of symptoms including dizziness and possibly loss of consciousness. To increase the rate of weight loss, respiration as a whole must be increased through physical activity in order to accelerate the biochemical break down of glycogen and fat molecules, and thus increase the rate of production carbon dioxide.

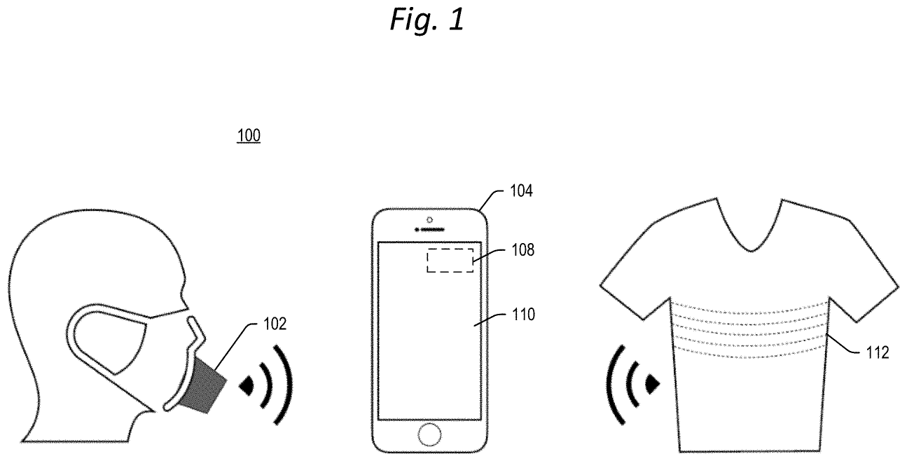

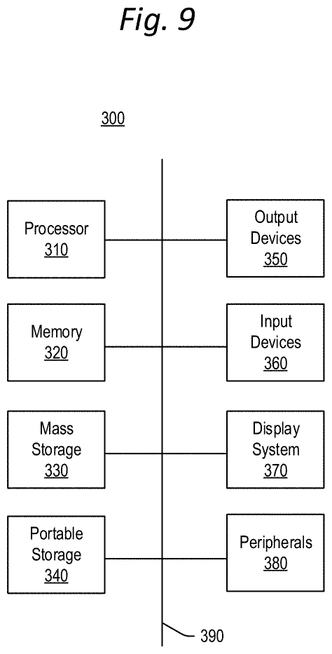

It is thus a feature of the present technology to monitor weight loss by measuring carbon mass lost through exhalation of carbon dioxide. Referring now to FIG. 1, the system 100 of the present technology accomplishes this through a capture device 102 for measuring exhaled carbon dioxide. As explained below with respect to FIG. 3, the capture device 102 may include a spirometer 126 for measuring a volume of each exhaled breath, and a capnometer 130 for measuring a concentration (for example by percentage) of carbon dioxide in the volume of exhaled breath. The breath volume and carbon dioxide concentration may be wirelessly communicated to a computing device 106. In embodiments, the device 106 may be a smart phone, but may be a smart watch, tablet, laptop, desktop or other computer. These computing devices may be stand-alone components, or they may be integrated into a home or office appliance or incorporated into an automobile. The computing device may include a processor 108 for executing an algorithm for converting the measured volume and concentration of carbon dioxide into a mass of exhaled carbon. The computing device may include a display 110 for displaying a user interface allowing users to easily see information relating to the mass of exhaled carbon. This user interface may include for example a waveform of exhaled carbon dioxide and other parameters relating to the mass of the exhaled carbon. Further details of a sample computing system are set forth below in FIG. 9.

The system 100 may further include a respiratory inductive plethysmograph (RIP), or other RSV, device 112 incorporated into apparel. ‘Apparel’ as used herein is broadly interpreted to include any apparel, garment or textile worn over or around the chest and/or abdomen of a user, including for example short or long sleeve shirt, tee shirt, tank top, sweatshirt, button down shirt, blouse, dress, vest, pajamas, robe, brazier and hospital gown. For the purposes of the present application, apparel also includes a band or strap worn around the chest and/or abdomen incorporating the RIP or other RSV device.

The RIP device 112 may be calibrated using the capnometer device 102 (also referred to herein as a capture device 102) and computing device 104. Thereafter, the RIP device 112 may be used without the capnography device 102 to measure exhaled carbon dioxide, which measurements are thereafter communicated to the computing device which processes the information and displays it in a user interface. While the following description refers to a RIP device, it is understood that the device 112 may be other RSV devices in further embodiments.

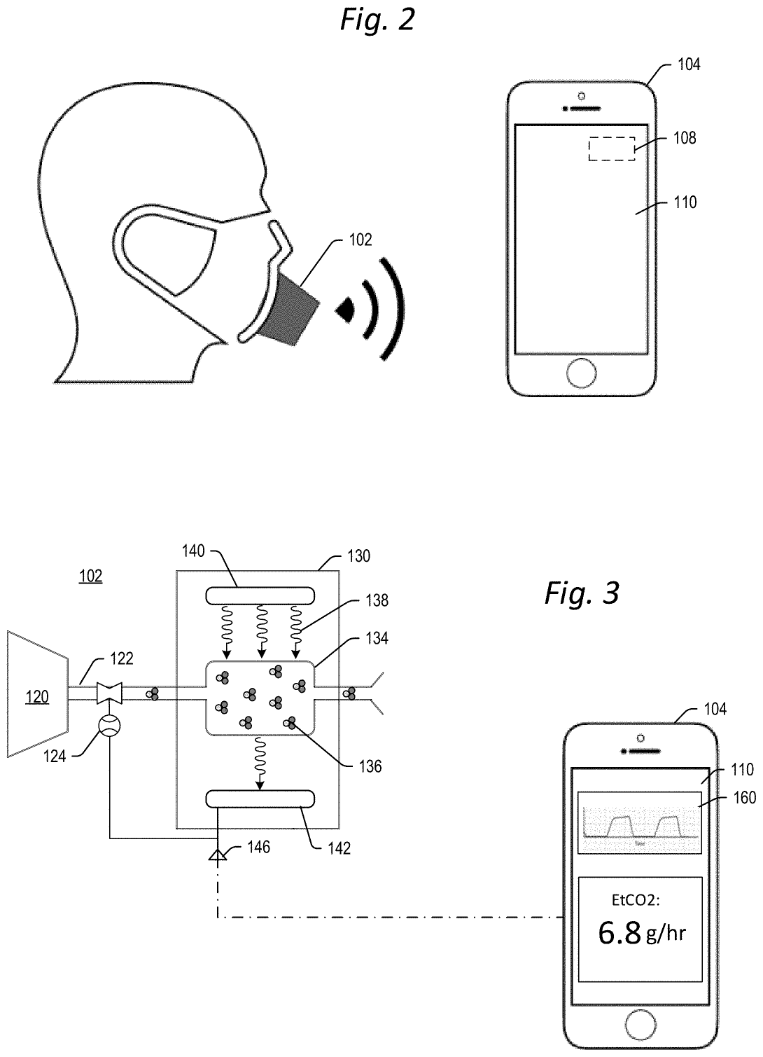

As indicated in FIG. 2, the present technology may use the capture device 102 with the computing device 104 alone (i.e., without the RIP device 112). Further details of the capture device 102 will now be explained with reference to FIG. 3. Carbon dioxide is a constant metabolic product of the body's cells, and it is transported through the blood system to the lungs, from which it is eliminated through the alveolar membrane. The capture device 102 includes a mask 120 fitting directly over the nose and/or mouth of a user, or a mouthpiece, to capture the full volume of exhaled carbon dioxide and other gasses of the user while breathing. The mask 120 is connected to a tube 122. A spirometer 126 (shown symbolically) may be provided in the flow path of the gas travelling through tube 122 to measure the volume of exhaled air (VE) on a breath-by-breath basis. Spirometer 126 may include one or more of a variety of different sensors for sensing flow rate over time of exhaled breath, including by pneumotachometer, pressure transducers and electronic or ultrasonic sensors. Other types of spirometers are possible.

The capture device 102 may further include a capnometer 130 for measuring carbon dioxide concentration in exhaled breath. Exhaled breath passes through tube 122, by or through the spirometer 126 and then through the capnometer 130. Thus, spirometer 126 is said to be upstream of the capnometer 130 along tube 122. In further embodiments, the spirometer 126 may alternatively be located downstream of the capnometer 130 along tube 122. In still further embodiments, the spirometer 126 and the capnometer 130 may be integrated together as a single unit.

The capnometer 130 measures the concentration of carbon dioxide exhaled out of the body throughout each breath cycle. This concentration of carbon dioxide is referred to as PCO₂. The capnometer 130 includes a chamber 134 which captures the full volume of breath from tube 122 as it is exhaled. Exhaled breath may be pushed into the chamber 134 by the force of the user's lungs. Exhaled breath then passes through the chamber 134 to the surrounding environment. Alternatively or additionally, a pump (not shown) may be included downstream of the chamber 134 to pull the volume of exhaled breath into the chamber 134. Moreover, in further embodiments, a vapor chamber may be provided upstream of the chamber 134 to remove vapor from the exhaled breath. Vapor may additionally or alternatively be removed by heating the tube 122 and/or chamber 134. The vapor chamber may be omitted in further embodiments.

Carbon dioxide molecules (136) are known to absorb infrared (IR) wavelengths (138) of a certain frequency. An emitter 140 is provided for emitting IR wavelengths at that frequency toward and through the chamber 134. The chamber 134 may be formed of a material such as sapphire that is transparent to infrared frequencies (or at least to those produced by emitter 140). The capnometer 130 further includes a sensor 142, on the opposed side of the chamber 134 from the emitter 140. The sensor 142, which may for example be a non-dispersive infrared (NDIR) sensor, receives the IR wavelengths 138 that are not absorbed by the carbon dioxide molecules 136 in chamber 134. The amount/intensity of the wavelengths 138 passing completely through the chamber 134 and incident on the sensor 142 will be inversely proportional to the end tidal carbon dioxide in the chamber 134. The sensor 142 converts this value to a voltage representative of the concentration by percentage of carbon dioxide, PCO₂. It is understood that other types of carbon dioxide sensors may be used.

The capture device 102 further includes a transmitter 146 for transmitting the total volume of exhaled carbon dioxide, VCO₂, measured by the spirometer 126. The sensor 142 of the capnometer 130 is also coupled to the transmitter 146, so that the concentration by percentage of carbon dioxide, PCO₂, is also sent to the computing device 104. The transmitter may work according to Bluetooth or WiFi specifications, or may be hardwired to the computing device.

Respiratory data may be captured by the capture device 102 at various time intervals, such as for example times per second, though other sampling rates are possible. The recorded respiratory data may be uploaded from the capture device 102 to locations in addition to the computing device 104, such as for example a central server of a service storing and monitoring carbon exhalation and weight loss for multiple users. The above description of the spirometer 126 and/or the capnometer 130 are provided by way of example only, and it is understood that spirometer 126 and/or capnography device 130 may include additional and/or alternative components in further embodiments.

In further embodiments, the capture device 102 may further include a known oxygen monitor (not shown) for monitoring a concentration by percentage of oxygen in an inhaled and exhaled breath. Thus, in addition to transmitting a volume of exhaled breath and the concentration of carbon dioxide therein, the capture device 102 may transmit a volume and concentration of inhaled and exhaled oxygen.

In accordance with further aspects of the present technology, the processor 108 of the computing device 104 executes a mass computation software algorithm which receives the raw respiratory data from the capture device, and uses that to determine an actual mass of exhaled carbon in a given period of time (e.g., per breath, per minute, per hour, per day, etc.). The mass computation software algorithm may run as a client application on computing device 104. In further embodiments, the mass computation application may alternatively or additionally run on a client server of a service for storing and monitoring carbon exhalation and weight loss for multiple users.

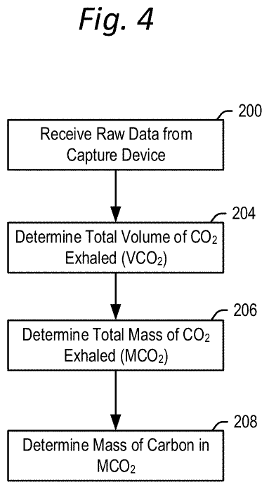

FIG. 4 is a flowchart of exemplary steps of the mass computation software algorithm according to embodiments of the present technology, executed for example on computing device 104. In step 200, the computing device 104 receives the raw respiratory data from the capture device 102. This data includes the volume of exhaled air, VE (from the spirometer), and the concentration by percentage of carbon dioxide, PCO₂, in the volume of exhaled air (from the capnometer device). This data may optionally further include the volume of inhaled air and the concentration of oxygen in the volume of inhaled and exhaled air.

In step 204, the volume of exhaled air VE is multiplied by the concentration of carbon dioxide PCO₂ in the exhaled air to determine the total volume of exhaled carbon dioxide, VCO₂. In step 206, the total volume of exhaled VCO₂ is multiplied by the density of carbon dioxide to provide the total mass of exhaled carbon dioxide, MCO₂. The density of carbon dioxide may be dynamically determined by computing device 104 using the given temperature and pressure at which the computing device 104 is being used. These values can be determined by other applications running on the computing device 104 and supplied to the mass computation algorithm. Alternatively, average assumed values for temperature and pressure may be used. Lastly, in step 208, the mass of exhaled carbon dioxide, MCO₂ is multiplied by a factor of 0.27291. This value represents the proportion of the mass of the carbon atoms in a carbon dioxide molecule. This results in the total mass of carbon in the exhaled breath. As noted, this value may be calculated multiple times during exhalation, or over some other time period.

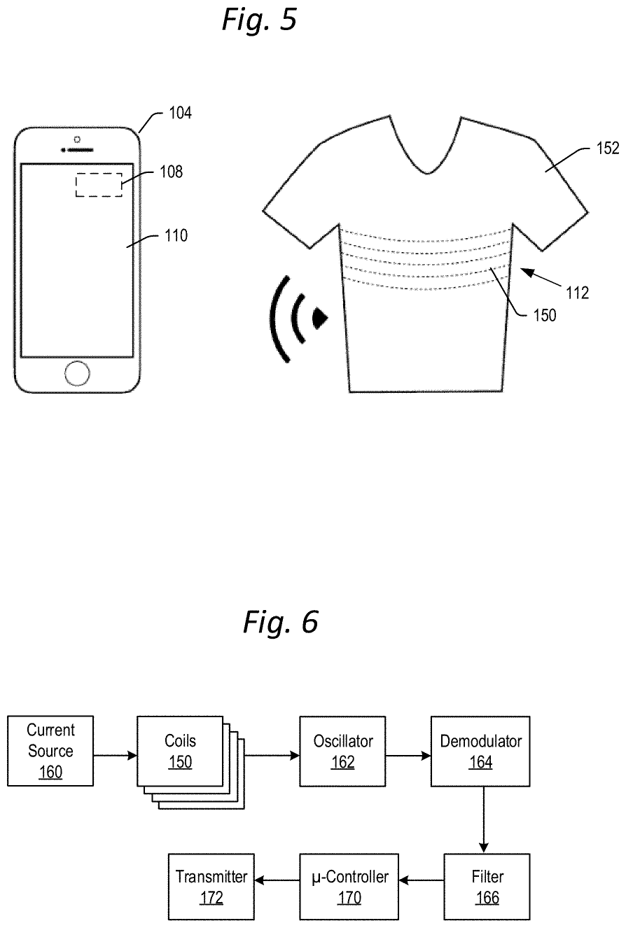

In further embodiments, the present technology comprises just the RIP device 112 working in cooperation with the computing device 104 as shown in FIG. 5. After calibration of the RIP device 112 by capture device 102 as explained below, the RIP device 112 may be used to provide exhaled carbon monitoring information without having to be tethered to the capture device 102.

Further details of RIP device 112 will now be explained with reference to FIGS. 5 and 6. Respiratory inductance plethysmography is a noninvasive method for indirectly measuring end tidal carbon dioxide exhaled as a function of measured volumetric changes in chest and/or abdomen volume. The RIP device 112 may comprise inductive sinusoidal (or other shaped) coils 150 incorporated into a flexible apparel 152 in the chest and/or abdomen area. Alternatively, the coils 150 may be incorporated into an elastic band or strap, worn by itself or which is in turn incorporated into an apparel in the chest and/or abdomen area. The apparel is preferably form fitting to lie in contact with the wearer's chest and/or abdomen.

In embodiments, the coils are positioned within apparel so as to be positioned around a person's torso when worn, in an area spanning from the chest down to the abdomen, to capture the total area of a person's torso that expands and contracts with breathing. Each coil 150 measures expansion/contraction independently of the other coils. In embodiments, there may be between two and ten coils 150, though there may be more or less in further embodiments. The coils may be grouped together, though they may be separated into two groups of coils in further embodiments, one around the chest and one around the abdomen.

FIG. 6 is a block diagram showing a sample circuit using feedback measured by the coils 150 of the RIP device 112. As noted, other RSV devices may be used instead of a RIP device. A constant current is provided to each of the coils 150 from a power source 160, such as battery pack also incorporated into a pocket of the apparel. During inspiration/exhalation, the volume of the rib cage and abdomen increases/decreases, causing the coils to expand/contract. Expansion and contraction of the coils alters the self-inductance of the coils. The current from the coils is fed to an oscillator 162. Variation in the inductance of the coils 150 results in a proportional variation in the frequency of the oscillator's sinusoidal output. An FM demodulator 164 is used to convert the variation in frequency into a voltage. The voltage signal is then passed to a filter 166 which filters and amplifies the voltage before it is fed to a microcontroller 170. The microcontroller can perform various functions including digitizing the received voltage, and transmitting the digitized voltage to the computing device 104 via transmitter 172. Transmitter 172 may operate according to Bluetooth or WiFi specifications.

Respiratory data may be measured by the RIP device 112 at various time intervals, such as for example ten times per second, though other sampling rates are possible. The recorded respiratory data may be uploaded from the RIP device 112 to locations in addition to the computing device 104, such as for example a central server of a service storing and monitoring carbon exhalation and weight loss for multiple users. The above description of the RIP device 112 is provided by way of example only, and it is understood that RIP device 112 may include additional and/or alternative components in further embodiments.

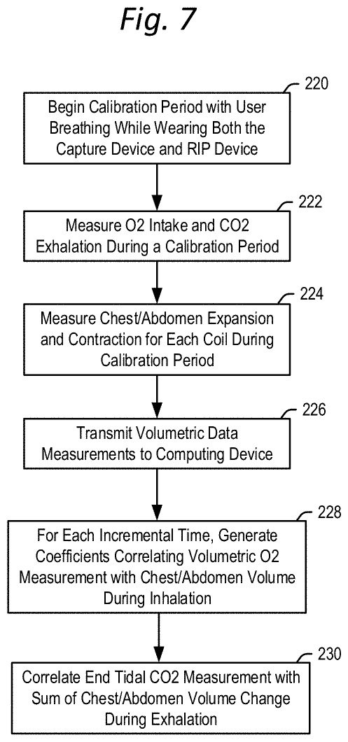

The RIP device 112 is capable of measuring volumetric changes in the chest and abdomen during breathing. These volumetric changes may be correlated to a volume of air inhaled and exhaled while breathing, via a calibration software algorithm run within the processor 108 of the computing device 104 as will now be explained with reference to the flowchart of FIG. 7.

The calibration process of the software algorithm begins in step 220 with the user breathing normally while wearing both the capture device 102 and RIP device 112. In step 222, the capture device 102 measures the volume of exhaled carbon dioxide, VCO₂, as explained above. In embodiments where the capture device 102 further includes an oxygen sensor, the capture device 102 may also measure the volume of inhaled and exhaled oxygen as explained above. In step 224, the RIP device 112 may measure the chest and/or abdomen contraction (and possibly expansion) volumes for each coil 150 as described above. In step 226, these measurements are transmitted to the computing device 104 as described above.

In step 228, the measured VCO₂ for a given instant in time is synchronized to the chest/abdomen volume as seen by each coil at the same instant in time. This effectively correlates chest/abdomen volume measured by the RIP device to a given volume VCO₂. From these correlations, the calibration algorithm is able to generate coefficients for each measured chest/abdomen volume, for each coil, that provide a volume of carbon dioxide, VCO₂, for chest/abdomen volumes measured by the different coils. The coefficients may be developed for each coil. In further embodiments, coefficients may be developed for the group of coils as a whole.

After the calibration process is complete, using the determined coefficients and the information received from the RIP device 112 alone, the computing device 104 is able to indirectly determine total volume of exhaled carbon dioxide, and the mass of carbon in the exhaled volume of carbon dioxide, as explained above. This offers potential advantages over the use of the capture device and mask, in that the user may wear the RIP device 112 to determine and monitor the amount of carbon exhaled while engaged in their normal day-to-day activities, including exercise and sleep.

During calibration, the software algorithm of the present technology compares simultaneous measurements made by the mask and sensors in the shirt or other apparel, adhesive straps or any other sensor used, in order to determine a range of coefficients by which to multiply the sensor measurements when apparel, strap or other sensor is worn in the absence of the mask. The software algorithm may be augmented by incorporating measurements from additional sensors, either integrated into sensors measuring breathing, or sensors in separate devices simultaneously worn by the user, such as a smart phone, smart watch, adhesive “bio button,” etc. For example, measurements from the following sensors obtained during the calibration process could potentially improve the accuracy and or precision of the software algorithm.
Heart Rate Monitor
Pulse Oximeter
Transcutaneous Carbon Dioxide sensor
Skin Temperature sensor, and/or
Accelerometers and/or gyroscopes.

While the wearable device works by respiratory inductive plethysmograph in the embodiments described above, it is understood that the wearable device may use other technologies to measure volumetric changes of the wearer's chest and/or abdomen in further embodiments. Such additional technologies are described for example in U.S. Patent Publication No. US20150289785A1, entitled, “Apparatus and Method for Monitoring Respiration Volumes and Synchronization of Triggering in Mechanical Ventilation by Measuring the Local Curvature of the Torso Surface,” published Oct. 15, 2015, which patent publication is incorporated by reference herein in its entirety.

Still further technologies may be used to measure volumetric changes the wearer's chest and/or abdomen in further embodiments. For example, in further embodiment, piezoresistive strain gauges worn as a band, or woven into the fabric of a garment or adhesive tape to measure the expansion and contraction of the chest and abdomen in multiple directions (horizontal, vertical, diagonal). The measurements from the piezoresistive strain gauges may be used for the input in determining the tidal volume of each breath.

In a further embodiment, potential dividers may be incorporated into a shirt or other textile. In this embodiment, conductive fibers may be incorporated into flexible fabric. The conductive fibers may be used to form a functional potential divider for the measurement of joint motion. This design could be adapted to measure the expansion and contraction of the chest and abdomen. A still further option are fiber-optic strain sensors incorporated into a shirt or other garment. Intensity-based and fiber Bragg grating sensors may be used for the measurement of strain, which strain measurement may then be used for the input in determining the tidal volume of each breath.

While embodiments of RIP device 112 above measure changes in strain caused by the expansion and contraction of the thorax and/or abdomen during the breathing cycle, other techniques may be used. For example, The capacitance of the human body changes throughout the breathing cycle due to the difference between the dielectric properties of the body tissues and of air. Electrodes worn on the chest or abdomen and in corresponding locations on the back can be used to measure the change in capacitance. These changes in capacitance may be used for the input in determining the tidal volume of each breath. Similarly, impedance across the thorax changes throughout the breathing cycle because i) increased gas volume in the chest decreases conductivity and ii) expansion of the chest increases the conductance path. These changes in impedance may be measured by passing a high frequency, low-amplitude alternating current between two or more electrodes worn on the chest, and the resulting impedance change measurement may be used for the input in determining the tidal volume of each breath.

In further embodiments, the movement of the chest wall may be measured and used as an input in determining the tidal volume of each breath. For example, accelerometers and/or gyroscopes can be used to measure movements of the chest wall and/or respiratory rates due to breathing. Non-respiratory motion can be filtered out. Magnetic field sensors can alternatively or additionally be used to measure movements of the chest wall by measuring either i) changes in the direction of magnetometer or ii) changes in the strength of the magnetic field of a permanent magnet worn by the user. Image recognition is another option. Image sensors, for example worn in the wristband of a watch, may be used as a sensor for measuring tidal volume.

Cardiac activity may further be used to generate input in determining the tidal volume of each breath. In one such example, electrocardiogram sensors may be worn or incorporated into apparel. Electrocardiogram (ECG) signals are affected by the motion of the electrodes relative to the heart due to respiratory and non-respiratory body movements and by changes in the impedance of the thoracic cavity due to breathing. ECG signals are also affected by the increase in heart rate during inhalation and decrease during the exhalation. These ECG signals may be measured by electrocardiogram sensors and used for the input in determining the tidal volume of each breath.

Photoplethysmography sensors are another option. Light emitting diodes combined with photodiodes worn on the fingertip, ear or toes (and possibly the wrist) can detect changes in blood volume and, therefore, heart cardiac activity. Breathing affects these signals by i) decreasing the blood stroke volume during inhalation due to changes in the intrathoracic pressure ii) increasing the heart rate during inhalation and decreasing heart rate during exhalation and iii) drift in the baseline signal due to changes in blood volume. The signals from photoplethysmography sensors may be used for the input in determining the tidal volume of each breath.

Another option for measuring tidal volume is to measure respiratory sounds. The frequency and amplitude of the sounds produced in the airways during inhalation and exhalation have been shown to have a direct relationship with airflow. Recording these sounds with microphones placed in in apparel or worn on one or more parts of the body could provide breath-by-breath measurement of the tidal volume.

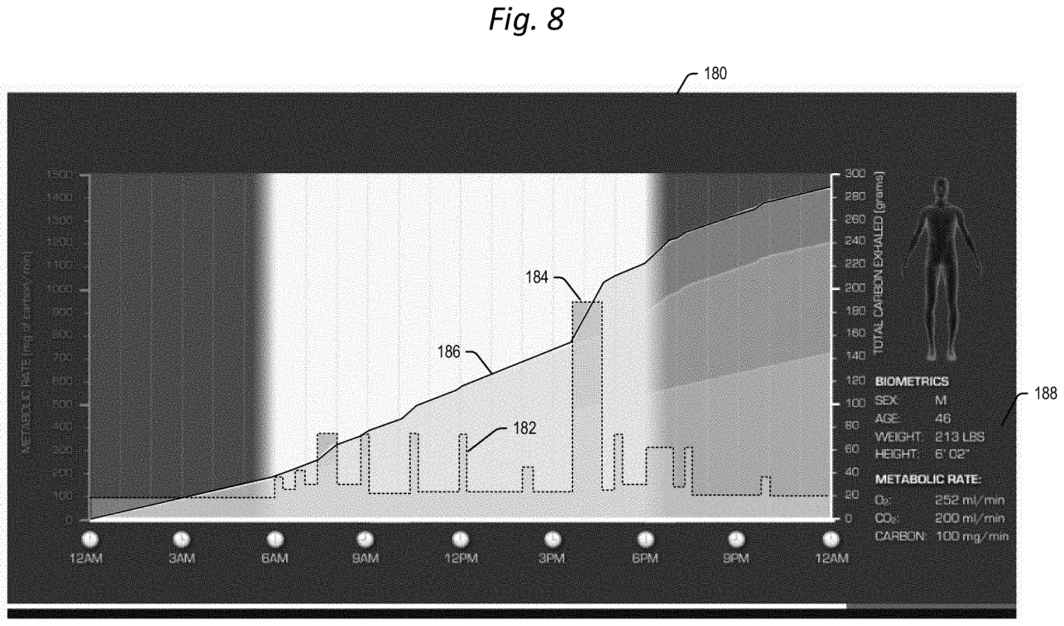

The computing device 104 may display a variety of information to the user related to the determined mass of expelled carbon over a given period of time. In FIG. 3, the display 110 shows a graphic of a waveform 160 showing the volume of carbon dioxide expelled in each breath (two breaths shown in the waveform graphic), as well as a display of the total mass of carbon exhaled (in grams per hour in this example). The displayed value may be updated multiple times per second or for each breath.

Where there is more screen real estate, such as for example on a tablet, laptop or other device having a larger display, the present technology may generate a user interface with more rich and detailed information. FIG. 8 shows one example of a user interface 180 that may be displayed by the present technology using a larger display 110.

The user interface 180 shows a vertical axis on the left including the metabolic rate at which carbon is expelled through exhalation over the course of a day (in mg/minute here). This amount is shown over time using a dashed line 182. As seen, low levels of carbon are given off during sleeping hours, but every day activities can increase respiration rates and increase the amount of carbon lost through exhalation. These every day activities may include any routine activities, such as getting out bed, making breakfast, walking to a bus, getting up from work to take a break, etc. The example user interface includes a large increase in carbon exhalation at 184, which may for example be exercise such as jogging, weight lifting or other work out or sporting activity, or strenuous work such as shoveling dirt, chopping wood, or moving heavy objects.

The user interface 180 shows a vertical axis on the right including the total cumulative mass of carbon expelled through exhalation over the course of a day (in grams here) for the user activity indicated by the dashed line. The total cumulative mass of carbon exhaled is shown by a solid line 186. As shown, increased user activity and metabolic rate at a given time results in a jump in the amount of carbon mass exhaled. The user interface may also display user information 188. The user interface 180 allows users to easily monitor their carbon loss through their daily activity, and provides a clear indication of which activities allow them to exhale the most carbon and lose the most weight per minute. In further embodiments, the user interface 180 could show a wide variety of other information, possibly customized by the user, including for example actual weight loss due to exhaled carbon.

FIG. 9 illustrates an exemplary computing system 300 that may be any embodiment of computing device 104 used to implement embodiments of the present technology. The computing system 300 of FIG. 9 includes one or more processors 310 and main memory 320. Processor 310 may for example be the processor 108 described above. Main memory 320 stores, in part, instructions and data for execution by processor unit 310. Main memory 320 can store the executable code when the computing system 300 is in operation. The computing system 300 of FIG. 9 may further include a mass storage device 330, portable storage medium drive(s) 340, output devices 350, user input devices 360, a display system 370, and other peripheral devices 380.

The components shown in FIG. 9 are depicted as being connected via a single bus 390. The components may be connected through one or more data transport means. Processor unit 310 and main memory 320 may be connected via a local microprocessor bus, and the mass storage device 330, peripheral device(s) 380, portable storage medium drive(s) 340, and display system 370 may be connected via one or more input/output (I/O) buses.

Mass storage device 330, which may be implemented with a magnetic disk drive an optical disk drive, non-volatile semiconductor memory, or other technologies, is a non-volatile storage device for storing data and instructions for use by processor unit 310. Mass storage device 330 can store the system software for implementing embodiments of the present invention for purposes of loading that software into main memory 320.

Portable storage medium drive(s) 340 operate in conjunction with a portable non-volatile storage medium, such as a floppy disk, compact disk or digital video disc, to input and output data and code to and from the computing system 300 of FIG. 9. The system software for implementing embodiments of the present invention may be stored on such a portable medium and input to the computing system 300 via the portable storage medium drive(s) 340.

Input devices 360 may provide a portion of a user interface. Input devices 360 may include an alpha-numeric keypad, such as a keyboard, for inputting alpha-numeric and other information, or a pointing device, such as a mouse, a trackball, stylus, or cursor direction keys. Additionally, the system 300 as shown in FIG. 9 includes output devices 350. Suitable output devices include speakers, printers, network interfaces, and monitors. Where computing system 300 is part of a mechanical client device, the output device 350 may further include servo controls for motors within the mechanical device.

Display system 370 may include a liquid crystal display (LCD) or other suitable display device. Display system 370 receives textual and graphical information and processes the information for output to the display device.

Peripheral device(s) 380 may include any type of computer support device to add additional functionality to the computing system. Peripheral device(s) 380 may include a modem or a router.

The components contained in the computing system 300 of FIG. 9 are those typically found in computing systems that may be suitable for use with embodiments of the present invention and are intended to represent a broad category of such computer components that are well known in the art. Thus, the computing system 300 of FIG. 9 can be a personal computer, hand held computing device, telephone, mobile computing device, workstation, server, minicomputer, mainframe computer, or any other computing device. The computer can also include different bus configurations, networked platforms, multi-processor platforms, etc. Various operating systems can be used including UNIX, Linux, Windows, Macintosh OS, Android, and other suitable operating systems.

Some of the above-described functions may be composed of instructions that are stored on storage media (e.g., computer-readable medium). The instructions may be retrieved and executed by the processor. Some examples of storage media are memory devices, tapes, punched cards, disks, and the like. The instructions are operational when executed by the processor to direct the processor to operate in accord with the invention. Those skilled in the art are familiar with instructions, processor(s), and storage media.

It is noteworthy that any hardware platform suitable for performing the processing described herein is suitable for use with the invention. The terms “computer-readable storage medium” and “computer-readable storage media” as used herein refer to any medium or media that participate in providing instructions to a CPU for execution. Such media can take many forms, including, but not limited to, non-volatile media and volatile media. Non-volatile media include, for example, optical or magnetic disks, such as a fixed disk. Volatile media include dynamic memory, such as system RAM. Common forms of computer-readable media include, for example, a floppy disk, a flexible disk, a hard disk, magnetic tape, any other magnetic medium, a CD-ROM disk, digital video disk (DVD), any other optical medium, any other physical medium with patterns of marks or holes, a RAM, a PROM, an EPROM, an EEPROM, a FLASHEPROM, any other memory chip or cartridge, or any other medium from which a computer can read.

Various forms of computer-readable media may be involved in carrying one or more sequences of one or more instructions to a CPU for execution. A bus carries the data to system RAM, from which a CPU retrieves and executes the instructions. The instructions received by system RAM can optionally be stored on a fixed disk either before or after execution by a CPU.

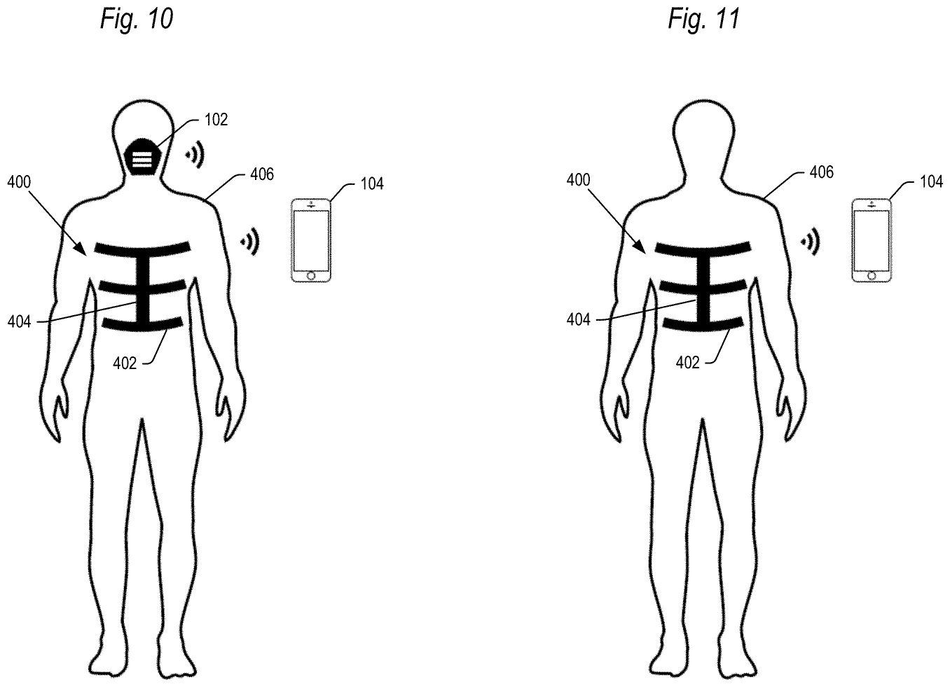

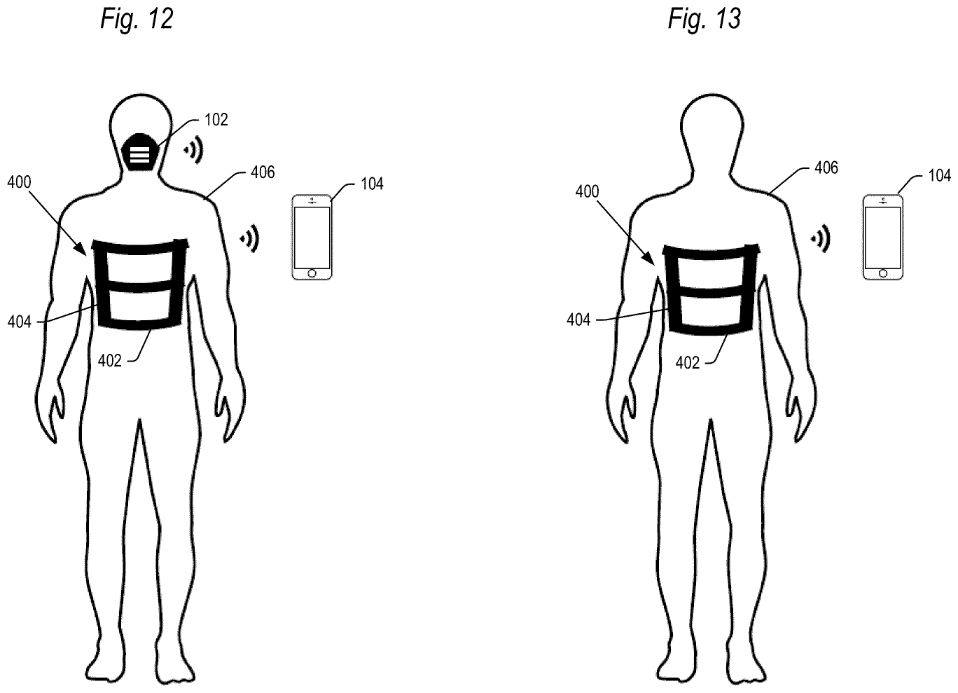

As noted above, RIP or other sensors may be incorporated into apparel, but as an alternative, the RIP or other sensors may be incorporated into adhesive or other straps worn directly on a user's torso or other body part. FIGS. 10 and 11 show a configuration of straps 400 which may be used. This embodiment shows three straight or curved horizontal strap segments 402 connected by a vertical strap segment 404. In FIG. 10, the straps 400 are worn by a user 406 during the calibration process with the capnography device 102. The information may be processed and calibrated by a computing device 104. In FIG. 11, the straps 400 are worn without the capnography device 102, with the computing device 104 using the calibration information to measure tidal volume. FIGS. 12 and 13 show an alternative configuration of straps 400 including three straight or curved horizontal strap segments 402 connected by a pair of vertical or slightly off-vertical strap segments 404. The tidal volume is calibrated in FIG. 12 using the capnography device 102, and measured in FIG. 13 using the computing device 104. The straps 400 may be worn on the chest and/or back. Other configurations of straps are contemplated.

In summary, the present technology relates to a system for monitoring respiration, comprising: a capture device, the capture device comprising a first device configured to detect a volume of air expelled during a breath, and a second device configured to detect a concentration of carbon dioxide in the breath; a device incorporated into apparel or to be worn by itself around a chest and/or abdomen, the device configured to detect volumetric changes in the chest and/or abdomen during breathing; and a computing device configured to execute a first algorithm to: receive the volume of air expelled during the breath from the capture device, receive the concentration of carbon dioxide in the breath from the capture device, and determine a mass of the carbon exhaled in the breath using the received volume of expelled air and concentration of carbon dioxide in the breath; and the computing device configured to execute a second algorithm to: receive the volume of air expelled during the breath from the capture device, receive the concentration of carbon dioxide in the breath from the capture device, receive the volumetric changes in the chest and/or abdomen during the breath, correlate a volumetric change in the chest and/or abdomen with an amount of air expelled during the breath, and determine a mass of the carbon exhaled in the breath using the correlated change in chest and/or abdomen volume and concentration of carbon dioxide in the breath.

In a further example, the present technology relates to a method of monitoring respiration, comprising: a) detecting a volume of air expelled during a breath using a first device; b) detecting a concentration of carbon dioxide in the breath using the first device; c) detecting volumetric changes in a chest and/or abdomen during breathing using a second device; d) determining a mass of carbon exhaled in the breath using the received volume of expelled air and concentration of carbon dioxide in the breath from the first device; e) calibrating the second device using and the received volume of expelled air as detected by the first device; and f) determining a mass of carbon exhaled in the breath using volumetric changes in a chest and/or abdomen during breathing from the second device after said step (e) of calibrating the second device.

For purposes of this document, a connection may be a direct connection or an indirect connection (e.g., via one or more other parts). In some cases, when an element is referred to as being connected, affixed or coupled to another element, the element may be directly connected to the other element or indirectly connected to the other element via intervening elements. When elements are referred to as being directly connected, directly affixed or directly coupled to each other, then there are no intervening elements between the directly connected, directly affixed or directly coupled elements.

The foregoing detailed description of the invention has been presented for purposes of illustration and description. It is not intended to be exhaustive or to limit the invention to the precise form disclosed. Many modifications and variations are possible in light of the above teaching. The described embodiments were chosen in order to best explain the principles of the invention and its practical application to thereby enable others skilled in the art to best utilize the invention in various embodiments and with various modifications as are suited to the particular use contemplated. It is intended that the scope of the invention be defined by the claims appended hereto.

What is claimed is:

1. A system for monitoring respiration, comprising:
a capture device, the capture device comprising a first device configured to detect a volume of air expelled during a breath, and a second device configured to detect a concentration of carbon dioxide in the breath;
a device incorporated into apparel or to be worn by itself around a chest and/or abdomen, the device configured to detect volumetric changes in the chest and/or abdomen during breathing; and
a computing device configured to execute a first algorithm to:
receive the volume of air expelled during the breath from the capture device,
receive the concentration of carbon dioxide in the breath from the capture device, and
determine a mass of the carbon exhaled in the breath using the received volume of expelled air and concentration of carbon dioxide in the breath; and
the computing device configured to execute a second algorithm to:
receive the volume of air expelled during the breath from the capture device,
receive the concentration of carbon dioxide in the breath from the capture device,
receive the volumetric changes in the chest and/or abdomen during the breath,
correlate a volumetric change in the chest and/or abdomen with an amount of air expelled during the breath, and
determine a mass of the carbon exhaled in the breath using the correlated change in chest and/or abdomen volume and concentration of carbon dioxide in the breath.

2. The system of claim 1, wherein the first device comprises a spirometer.

3. The system of claim 1, wherein the second device comprises a capnometer.

4. The system of claim 1, wherein the device incorporated into apparel or to be worn by itself around a chest and/or abdomen comprises a respiratory volume sensor.

5. The system of claim 1, wherein the computing device comprises at least one of a smart phone, a smart watch, tablet, laptop or desktop computer.

6. The system of claim 5, wherein the computing device is integrated into a home or office appliance or incorporated into an automobile.

7. The system of claim 1, wherein the computing device is further configured to present a user interface presenting information relating to the mass of carbon exhaled.

8. A method of monitoring respiration, comprising:
a) detecting a volume of air expelled during a breath using a first device;
b) detecting a concentration of carbon dioxide in the breath using the first device;
c) detecting volumetric changes in a chest and/or abdomen during breathing using a second device;
d) determining a mass of carbon exhaled in the breath using the received volume of expelled air and concentration of carbon dioxide in the breath from the first device;
e) calibrating the second device using and the received volume of expelled air as detected by the first device; and
f) determining a mass of carbon exhaled in the breath using volumetric changes in a chest and/or abdomen during breathing from the second device after said step (e) of calibrating the second device.

9. The method of claim 8, wherein said step a) of detecting a volume of air expelled during a breath comprises detecting a flow rate of air over a given period of time.

10. The method of claim 8, wherein said step b) of detecting a concentration of carbon dioxide in the breath comprises radiating a chamber of carbon dioxide molecules with an infrared wavelength absorbed by carbon dioxide molecules and detecting the amount of infrared wavelengths passing through the chamber.

11. The method of claim 8, wherein said step c) of detecting volumetric changes in a chest and/or abdomen during breathing comprises the step of detecting a change in volume of the chest and/or abdomen.

12. The method of claim 8, wherein said step d) of determining a mass of carbon exhaled in the breath comprises the steps of:
determining a total volume of carbon dioxide from the total volume of the breath and the concentration of carbon dioxide in the breath;
determining a total mass of carbon dioxide in the total volume of carbon dioxide using a density of carbon dioxide; and
determining a total mass of carbon in the exhaled breath using a percentage of the mass of carbon in the mass of a carbon dioxide molecule.

13. The method of claim 12, wherein said step of determining a total mass of carbon dioxide in the total volume of carbon dioxide using a density of carbon dioxide comprises the step of determining a temperature and pressure at which said step d) of determining the mass of carbon is performed.

14. The method of claim 12, wherein said step of determining a total mass of carbon dioxide in the total volume of carbon dioxide using a density of carbon dioxide comprises the step of using an average assumed temperature and pressure.

15. The method of claim 8, further comprising the step of displaying information relating to the mass of carbon in the exhaled breath.
